(19) United States
(12) Patent Application Publication
(10) Pub. No.: US 2020/0229789 A1
(43) Pub. Date: Jul. 23, 2020

(54) ULTRASOUND DIAGNOSTIC APPARATUS AND METHOD FOR CONTROLLING ULTRASOUND DIAGNOSTIC APPARATUS

(71) Applicant: FUJIFILM Corporation, Tokyo (JP)

(72) Inventors: Katsuya YAMAMOTO, Kanagawa (JP); Kaku IRISAWA, Kanagawa (JP)

(73) Assignee: FUJIFILM Corporation, Tokyo (JP)

(21) Appl. No.: 16/843,646

(22) Filed: Apr. 8, 2020

Related U.S. Application Data
(63) Continuation of application No. PCT/JP2018/038810, filed on Oct. 18, 2018.

(30) Foreign Application Priority Data
Dec. 6, 2017 (JP) 2017-234340

(51) Int. Cl. A61B 8/12; A61B 8/00; A61B 90/00

(52) U.S. Cl. CPC A61B 8/12; A61B 8/4483; A61B 8/54; A61B 8/4281; A61B 2090/062; A61B 90/03; A61B 90/06; A61B 2090/033; A61B 8/565

(57) ABSTRACT

An ultrasound diagnostic apparatus includes a probe having an array transducer, a transmitting unit that transmits an ultrasound beam from the array transducer, an insertion device insertable into a subject and having a photoacoustic wave generation unit, a light source that irradiates the photoacoustic wave generation unit with light to generate a photoacoustic wave, a sequence control unit that performs control so that a photoacoustic wave is received each time the array transducer receives an ultrasound echo along a predetermined number of scan lines, an insertion depth detection unit that detects an insertion depth of the insertion device on the basis of a photoacoustic wave reception signal, and a notification unit that provides a notification to a user in a case where the insertion depth of the insertion device is deeper than a determined depth.

### CROSS-REFERENCE TO RELATED APPLICATIONS

This application is a Continuation of PCT International Application No. PCT/JP2018/038810 filed on Oct. 18, 2018, which claims priority under 35 U.S.C § 119(a) to Japanese Patent Application No. 2017-234340 filed on Dec. 6, 2017. Each of the above application(s) is hereby expressly incorporated by reference, in its entirety, into the present application.

### BACKGROUND OF THE INVENTION

### 1. Field of the Invention

The present invention relates to an ultrasound diagnostic apparatus and a method for controlling the ultrasound diagnostic apparatus, and more specifically to an ultrasound diagnostic apparatus including an insertion device such as a puncture needle, and a method for controlling the ultrasound diagnostic apparatus.

### 2. Description of the Related Art

Ultrasound diagnostic apparatuses are known in the related art as apparatuses for obtaining an image of the inside of a subject. An ultrasound diagnostic apparatus typically transmits ultrasound beams to a subject from an array transducer in which a plurality of elements are arrayed, and receives ultrasound echoes from the subject at the array transducer to acquire element data. Further, the ultrasound diagnostic apparatus is capable of electrically processing the acquired element data and generating an ultrasound image that shows a site of the subject.

In the related art, furthermore, an insertion device such as a puncture needle is inserted into a subject to perform treatment such as sampling and medication injection. In this manner, in a case where an insertion device is used to perform treatment such as sampling and medication injection, various contrivances are made so that the position of the tip portion of the insertion device can be determined for safety of the subject.

For example, JP2016-540604A discloses a system for detecting the position of an insertion device inserted into a subject using an ultrasound sensor embedded in the vicinity of the distal end of the insertion device and displaying the position of the tip portion of the insertion device in a generated ultrasound image.

JP2004-230182A discloses an ultrasound diagnostic apparatus that performs image processing on a generated ultrasound image to detect the position of an insertion device inserted into a subject. It is also disclosed in JP2004-230182A that Doppler signals are sequentially detected to detect the position of a blood vessel and that advancement of the insertion device is automatically stopped in a case where the insertion device and the blood vessel are close to each other.

### SUMMARY OF THE INVENTION

In the technique disclosed in JP2016-540604A, however, for example, the user needs to visually check the ultrasound image and to determine the position of the insertion device so as not to allow the insertion device to approach sites through which advancement of the insertion device is unwanted, such as blood vessels. In this manner, the grasp of the position of the insertion device by using the technique disclosed in JP2016-540604A largely depends on the user's attention, and there is thus a problem in that the risk of the insertion device touching sites such as blood vessels is high.

In the technique disclosed in JP2004-230182A, it is possible to automatically stop advancement of an insertion device inserted into a subject. However, it is necessary to constantly detect Doppler signals. In this manner, in a case where Doppler signals are constantly detected, the frame rate of an ultrasound image to be generated is typically slow, and a certain amount of time is required until the ultrasound diagnostic apparatus of JP2004-230182A determines that the insertion device has approached a blood vessel and advancement of the insertion device is stopped. There is thus a problem in that the risk of the insertion device touching the site, such as a blood vessel, is high.

In both the techniques disclosed in JP2016-540604A and JP2004-230182A, a user is not able to determine the position of the insertion device until an ultrasound image of one frame is generated. For this reason, a time lag may occur between the position of the insertion device determined by the user in the ultrasound image and the actual position of the insertion device. Due the time lag, the user may not be able to immediately stop advancement of the insertion device to avoid the insertion device touching a site such as a blood vessel.

The present invention has been made to solve the problems of the related art, and it is an object of the present invention to provide an ultrasound diagnostic apparatus that detects an insertion depth of an insertion device and that allows a user to take measures in a case where the insertion device approaches a site through which advancement of the insertion device is unwanted and to provide a method for controlling the ultrasound diagnostic apparatus.

To achieve the object described above, an ultrasound diagnostic apparatus of the present invention includes a probe having an array transducer, a transmitting unit that transmits respective ultrasound beams from the array transducer to a subject along a plurality of scan lines, an ultrasound image generation unit that converts ultrasound reception signals obtained from the array transducer in response to receipt of ultrasound echoes from the subject into an image to generate an ultrasound image of the subject, an insertion device insertable into the subject and having a photoacoustic wave generation unit, a light source that irradiates the photoacoustic wave generation unit of the insertion device with light to generate a photoacoustic wave from the photoacoustic wave generation unit, a sequence control unit that controls the transmitting unit and the light source so that the photoacoustic wave is received by the array transducer each time the array transducer receives an ultrasound echo along a predetermined number of scan lines, an insertion depth detection unit that detects an insertion depth of the insertion device on the basis of a photoacoustic wave reception signal obtained by the array transducer, and a notification unit that provides a notification to a user in a case where the insertion depth of the insertion device detected by the insertion depth detection unit is deeper than a determined depth.

The sequence control unit can control the transmitting unit and the light source so that the photoacoustic wave is received by the array transducer each time reception of an ultrasound echo along a single scan line is performed.

Alternatively, the sequence control unit can control the transmitting unit and the light source so that the photoacoustic wave is received by the array transducer each time reception of ultrasound echoes along a plurality of scan lines is performed.

Further, the ultrasound diagnostic apparatus can further include a depth setting unit that sets the determined depth.

In addition, the ultrasound diagnostic apparatus can further include an operating unit through which the user performs an input operation, and the depth setting unit can set, as the determined depth, a depth input by the user through the operating unit.

More specifically, the depth setting unit can set, as the determined depth, a depth at a position designated by the user through the operating unit in the ultrasound image.

Further, the ultrasound diagnostic apparatus can further include an image analysis unit that performs image analysis on the ultrasound image to detect an inhibition site into which an entry of the insertion device is inhibited.

In addition, the ultrasound diagnostic apparatus can further include an operating unit through which the user performs an input operation, and a depth candidate presentation unit that presents a plurality of depth candidates related to the determined depth to the user on the basis of the inhibition site detected by the image analysis unit, and the depth setting unit can set the determined depth from a depth candidate selected by the user from among the plurality of depth candidates through the operating unit.

Further, the depth setting unit can also set, as the determined depth, a depth at a shallowest position within an area occupied by the inhibition site detected by the image analysis unit.

Further, the ultrasound diagnostic apparatus can further include an ultrasound image update unit that updates the ultrasound image each time reception of an ultrasound echo along a predetermined number of scan lines is performed by the array transducer, and the image analysis unit can also detect the inhibition site in the ultrasound image updated by the ultrasound image update unit.

In addition, the ultrasound diagnostic apparatus can further include a depth update unit that updates the determined depth on the basis of an area occupied by the inhibition site detected by the image analysis unit each time the ultrasound image is updated by the ultrasound image update unit.

Further, the sequence control unit can control the transmitting unit to perform a pre-scan of the subject, and the image analysis unit can perform the image analysis on the ultrasound image obtained by the pre-scan.

The notification unit can provide the notification to the user by at least one of generating a warning sound or vibrating the probe.

Alternatively, the ultrasound diagnostic apparatus can further include a display unit that displays the ultrasound image, and the notification unit can provide the notification to the user by providing a warning display on the display unit.

In addition, the notification unit can provide the warning display by changing a color of a tip portion of the insertion device to be displayed on the display unit in accordance with a difference between the insertion depth of the insertion device and the determined depth.

Alternatively, the ultrasound diagnostic apparatus can further include a display unit that displays the ultrasound image, and the notification unit can provide the notification to the user by freezing the ultrasound image on the display unit.

Further, the insertion device can be a puncture needle, a catheter, or forceps.

A method for controlling an ultrasound diagnostic apparatus according to the present invention includes transmitting and receiving respective ultrasound beams to and from a subject along a plurality of scan lines, emitting light to an insertion device insertable into the subject and having a photoacoustic wave generation unit, receiving a photoacoustic wave generated from the photoacoustic wave generation unit in response to the photoacoustic wave generation unit being irradiated with the emitted light, controlling transmission and reception of the ultrasound beams, emission of light to the insertion device, and reception of the photoacoustic wave so that the photoacoustic wave is received each time an ultrasound echo is received along a predetermined number of scan lines, detecting an insertion depth of the insertion device on the basis of a signal of the photoacoustic wave that is received, and providing a notification to a user in a case where the detected insertion depth of the insertion device is deeper than a determined depth.

According to the present invention, an ultrasound diagnostic apparatus includes a sequence control unit that controls a transmitting unit, a receiving unit, and a light source so that a photoacoustic wave is received by an array transducer each time the array transducer receives an ultrasound echo along a predetermined number of scan lines, an insertion depth detection unit that detects an insertion depth of an insertion device on the basis of a signal of the photoacoustic wave that is received by the array transducer, and a notification unit that provides a notification to a user in a case where the insertion depth of the insertion device detected by the insertion depth detection unit is deeper than a determined depth. This enables rapid detection of the insertion depth of the insertion device and enables the user to immediately take measures in a case where the insertion device approaches a site through which advancement of the insertion device is unwanted.

### BRIEF DESCRIPTION OF THE DRAWINGS

FIG. 1 is a block diagram illustrating a configuration of an ultrasound diagnostic apparatus according to Embodiment 1 of the present invention;

FIG. 2 is a diagram illustrating an example of an insertion device in Embodiment 1 of the present invention;

FIG. 3 is a block diagram illustrating an internal configuration of a light source in Embodiment 1 of the present invention;

FIG. 4 is a block diagram illustrating an internal configuration of a receiving unit in Embodiment 1 of the present invention;

FIG. 5 is a block diagram illustrating an internal configuration of an ultrasound image generation unit in Embodiment 1 of the present invention;

FIG. 6 illustrates an example display of an ultrasound image in Embodiment 1 of the present invention;

FIG. 7 is a flowchart illustrating the operation of the ultrasound diagnostic apparatus according to Embodiment 1 of the present invention;

FIG. 8 is a conceptual diagram illustrating the timing of transmitting an ultrasound wave, the timing of receiving an ultrasound echo, a light emission timing, and a timing of receiving a photoacoustic wave in Embodiment 1 of the present invention;

FIG. 9 is a conceptual diagram illustrating how the insertion device is sensed in Embodiment 1 of the present invention;

FIG. 10 is a conceptual diagram illustrating a case where the insertion depth of the insertion device is deeper than a limit depth in Embodiment 1 of the present invention;

FIG. 11 is a flowchart illustrating the operation of an ultrasound diagnostic apparatus according to Embodiment 2 of the present invention;

FIG. 12 is a conceptual diagram illustrating the timing of transmitting an ultrasound wave, the timing of receiving an ultrasound echo, a light emission timing, and a timing of receiving a photoacoustic wave in Embodiment 2 of the present invention;

FIG. 13 is a block diagram illustrating a configuration of an ultrasound diagnostic apparatus according to Embodiment 3 of the present invention;

FIG. 14 is a block diagram illustrating an internal configuration of an ultrasound image generation unit in Embodiment 3 of the present invention;

FIG. 15 is a flowchart illustrating a limit depth setting operation in Embodiment 3 of the present invention;

FIG. 16 illustrates an example display of limit depth candidates in Embodiment 3 of the present invention;

FIG. 17 is a flowchart illustrating a limit depth setting operation in Embodiment 4 of the present invention;

FIG. 18 illustrates an example display of limit depth candidates in Embodiment 4 of the present invention;

FIG. 19 is a block diagram illustrating a configuration of an ultrasound diagnostic apparatus according to Embodiment 5 of the present invention;

FIG. 20 illustrates an example display of an automatically determined limit depth in Embodiment 5 of the present invention;

FIG. 21 illustrates another example display of the automatically determined limit depth in Embodiment 5 of the present invention;

FIG. 22 is a block diagram illustrating a configuration of an ultrasound diagnostic apparatus according to Embodiment 6 of the present invention;

FIG. 23 is a flowchart illustrating the operation of the ultrasound diagnostic apparatus according to Embodiment 6 of the present invention; and

FIG. 24 is a conceptual diagram illustrating the time point at which an ultrasound image is updated in Embodiment 6 of the present invention.

### DESCRIPTION OF THE PREFERRED EMBODIMENTS

The following describes embodiments of this invention with reference to the accompanying drawings.

### Embodiment 1

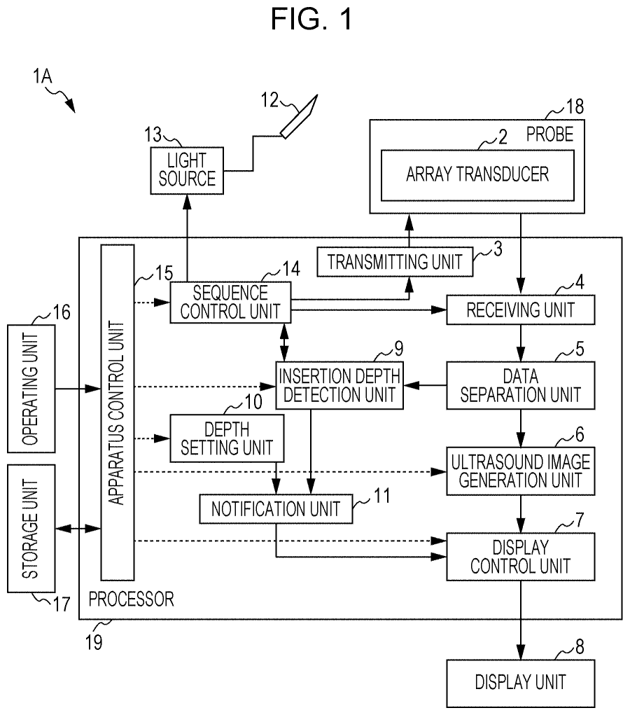

FIG. 1 illustrates a configuration of an ultrasound diagnostic apparatus 1A according to Embodiment 1 of the present invention. As illustrated in FIG. 1, the ultrasound diagnostic apparatus 1A includes an array transducer 2, and a transmitting unit 3 and a receiving unit 4 are connected to the array transducer 2. A data separation unit 5, an ultrasound image generation unit 6, a display control unit 7, and a display unit 8 are sequentially connected to the receiving unit 4. An insertion depth detection unit 9 is connected to the data separation unit 5, and a notification unit 11 is connected to the insertion depth detection unit 9. A depth setting unit 10 and the display control unit 7 are connected to the notification unit 11. The ultrasound diagnostic apparatus 1A includes an insertion device 12, and the insertion device 12 is connected to a light source 13. A sequence control unit 14 is connected to the transmitting unit 3, the receiving unit 4, the insertion depth detection unit 9, and the light source 13, and the insertion depth detection unit 9 and the sequence control unit 14 are connected to each other so as to enable two-way delivery of information.

Further, an apparatus control unit 15 is connected to the ultrasound image generation unit 6, the display control unit 7, the insertion depth detection unit 9, the depth setting unit 10, and the sequence control unit 14, and an operating unit 16 and a storage unit 17 are connected to the apparatus control unit 15. The apparatus control unit 15 and the storage unit 17 are connected to each other so as to enable two-way delivery of information.

The array transducer 2 is included in a probe 18, and the transmitting unit 3, the receiving unit 4, the data separation unit 5, the ultrasound image generation unit 6, the display control unit 7, the insertion depth detection unit 9, the depth setting unit 10, the notification unit 11, the sequence control unit 14, and the apparatus control unit 15 constitute a processor 19.

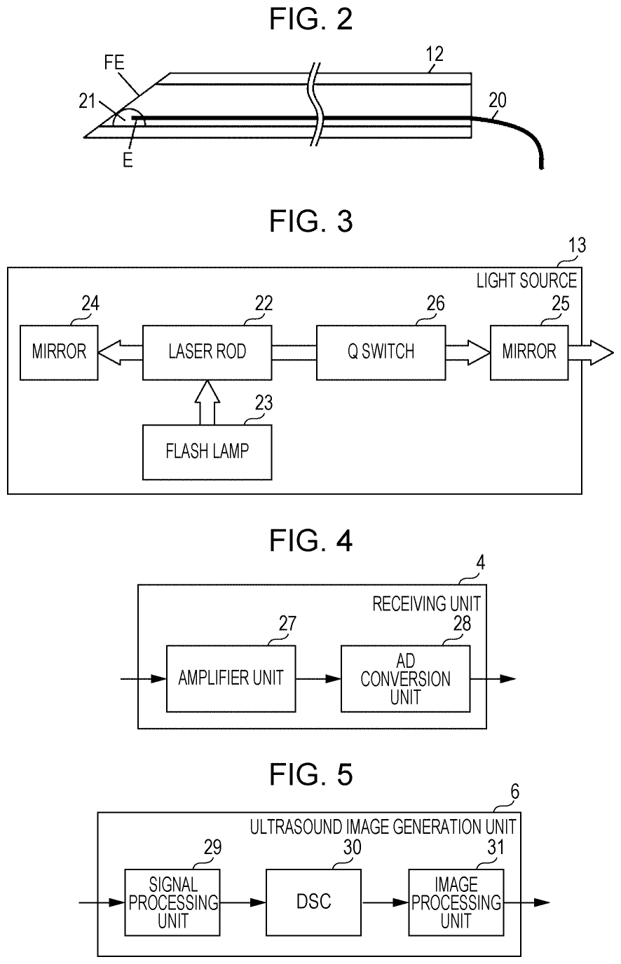

The insertion device 12 illustrated in FIG. 1 is inserted into a subject for ultrasound diagnosis and is used to perform treatment such as sampling and medication injection. The insertion device 12, examples of which can include a puncture needle, a catheter, and forceps, is implemented using a puncture needle illustrated in FIG. 2, for example. The insertion device 12 illustrated in FIG. 2 has provided therein a light guide member 20 such as an optical fiber that extends from the light source 13, which is externally arranged, to the vicinity of a tip portion FE of the insertion device 12. Further, a photoacoustic wave generation unit 21 is arranged inside the insertion device 12 in the vicinity of the tip portion FE of the insertion device 12, and a tip portion E of the light guide member 20 is embedded in the photoacoustic wave generation unit 21.

The photoacoustic wave generation unit 21 is composed of a light-absorbing material, for example, a synthetic resin such as an epoxy resin with a black pigment, a fluororesin, or a polyurethane resin. In a case where irradiated with light, the photoacoustic wave generation unit 21 contracts and expands to generate a photoacoustic wave. In the insertion device 12 illustrated in FIG. 2, in a case where the photoacoustic wave generation unit 21 is irradiated with light emitted from the light source 13 via the light guide member 20, a photoacoustic wave is generated from the photoacoustic wave generation unit 21.

As illustrated in FIG. 3, the light source 13 has a laser rod 22, a flash lamp 23, a mirror 24, a mirror 25, and a Q switch 26. The laser rod 22 is a laser medium. The laser rod 22 can be made of, for example, alexandrite crystal. The flash lamp 23 is an excitation light source and irradiates the laser rod 22 with excitation light. The excitation light source is not limited to the flash lamp 23, and a light source other than the flash lamp 23 may instead be used as an excitation light source.

The mirrors 24 and 25 face each other with the laser rod 22 interposed therebetween. The mirrors 24 and 25 form an optical resonator. In the optical resonator, the mirror 25 corresponds to the output side. The Q switch 26 is inserted into the optical resonator. The Q switch 26 can rapidly change from high insertion loss state to low insertion loss state within the optical resonator, thereby obtaining pulsed laser light. The pulsed laser light emitted from the output-side mirror 25 of the light source 13 is guided to the insertion device 12 through the light guide member 20.

The array transducer 2 of the probe 18 illustrated in FIG. 1 has a plurality of elements (ultrasound vibrators) that are arrayed one-dimensionally or two-dimensionally. Each of these elements transmits an ultrasound wave in accordance with a drive signal supplied from the transmitting unit 3 and outputs an ultrasound reception signal upon receipt of a reflected wave from the subject. Further, these elements receive a photoacoustic wave generated by the irradiation of the photoacoustic wave generation unit 21 of the insertion device 12 with light from the light source 13, and output photoacoustic wave reception signals.

Each element is configured using, for example, a vibrator produced by forming electrodes at both ends of a piezoelectric body composed of a piezoelectric ceramic typified by PZT (Lead Zirconate Titanate), a polymeric piezoelectric element typified by PVDF (Poly Vinylidene Di Fluoride), a piezoelectric single crystal typified by PMN-PT (Lead Magnesium Niobate-Lead Titanate), or the like.

The transmitting unit 3 of the processor 19 includes, for example, a plurality of pulse generators, and supplies to the plurality of elements respective drive signals whose amounts of delay are adjusted so that the ultrasound waves transmitted from the plurality of elements of the array transducer 2 form an ultrasound beam on the basis of a transmission delay pattern selected in accordance with a control signal from the sequence control unit 14. In this manner, in a case where a pulsed or continuous-wave voltage is applied to the electrodes of the elements of the array transducer 2, the piezoelectric bodies expand and contract. Pulsed or continuous-wave ultrasound waves are generated from the respective vibrators, and a composite wave of these ultrasound waves forms an ultrasound beam.

The transmitted ultrasound beam is reflected from, for example, a target such as a site of the subject and propagates toward the array transducer 2 of the probe 18. The ultrasound echo propagating toward the array transducer 2 in this manner is received by the respective elements of the array transducer 2. At this time, upon receipt of the propagating ultrasound echo, the respective elements of the array transducer 2 expand and contract to generate electrical signals, and these electrical signals are output to the receiving unit 4 as ultrasound reception signals.

The photoacoustic wave generated by the irradiation of the photoacoustic wave generation unit 21 of the insertion device 12 with the light emitted from the light source 13 is also received by the respective elements of the array transducer 2. At this time, upon receipt of the photoacoustic wave, the respective elements of the array transducer 2 expand and contract to generate electrical signals in a manner similar to that upon receipt of the ultrasound wave, and these electrical signals are output to the receiving unit 4 as photoacoustic wave reception signals.

The receiving unit 4 of the processor 19 performs processing of the ultrasound reception signals and processing of the photoacoustic wave reception signals output from the array transducer 2 in accordance with a control signal from the sequence control unit 14. As illustrated in FIG. 4, the receiving unit 4 has a configuration in which an amplifier unit 27 and an AD (Analog Digital) conversion unit 28 are connected in series. The amplifier unit 27 amplifies the ultrasound reception signals and the photoacoustic wave reception signals input from the respective elements of the array transducer 2 and transmits the amplified reception signals to the AD conversion unit 28. The AD conversion unit 28 converts the ultrasound reception signals and the photoacoustic wave reception signals transmitted from the amplifier unit 27 into digital data and sends the data to the data separation unit 5 of the processor 19.

The data separation unit 5 of the processor 19 separates the data of the ultrasound reception signals and the data of the photoacoustic wave reception signals output from the receiving unit 4, and outputs the data of the ultrasound reception signals to the ultrasound image generation unit 6 and the data of the photoacoustic wave reception signals to the insertion depth detection unit 9.

As illustrated in FIG. 5, the ultrasound image generation unit 6 of the processor 19 has a configuration in which a signal processing unit 29, a DSC (Digital Scan Converter) 30, and an image processing unit 31 are connected in series. The signal processing unit 29 performs reception focus processing in which the pieces of data of the ultrasound reception signals according to set sound velocities are given respective delays on the basis of a reception delay pattern selected in accordance with a control signal from the apparatus control unit 15 and are added together (phasing addition). Through the reception focus processing, a sound ray signal in which the focus of the ultrasound echo is narrowed to a single scan line is generated. Further, the signal processing unit 29 corrects the generated sound ray signal for attenuation caused by the propagation distance in accordance with the depth of the position at which the ultrasound wave is reflected, and then performs envelope detection processing to generate a B-mode image signal that is a tomographic image information related to tissue in the subject. The B-mode image signal generated in this way is output to the DSC 30.

The DSC 30 of the ultrasound image generation unit 6 performs raster conversion to convert the B-mode image signal into an image signal based on a typical television signal scanning method. The image processing unit 31 of the ultrasound image generation unit 6 performs various necessary image processing operations, such as brightness correction, gradation correction, sharpness correction, and color correction, on the image data obtained by the DSC 30, and then outputs the B-mode image signal to the display control unit 7.

The sequence control unit 14 of the processor 19 controls the transmitting unit 3, the receiving unit 4, and the light source 13 to control the timing of transmitting an ultrasound wave, the timing of starting an ultrasound echo receiving operation, the timing of emitting light from the light source 13, and the timing of starting a photoacoustic wave receiving operation. In the present invention, receiving a photoacoustic wave while generating an ultrasound image enables more rapid detection of the insertion depth of the insertion device 12. The timing of transmitting an ultrasound wave, the timing of starting an ultrasound echo receiving operation, the timing of emitting light from the light source 13, and the timing of starting a photoacoustic wave receiving operation in Embodiment 1 will be described in detail below.

Further, the sequence control unit 14 outputs the timing of starting a photoacoustic wave receiving operation by the receiving unit 4 via the array transducer 2 to the insertion depth detection unit 9 so that the insertion depth detection unit 9 can detect the insertion depth of the insertion device 12.

The insertion depth detection unit 9 of the processor 19 performs reception focus processing on the data of the photoacoustic wave reception signals output from the data separation unit 5 to generate a signal in which the focus of the photoacoustic wave is narrowed to a single scan line. Further, the insertion depth detection unit 9 detects the insertion depth of the insertion device 12 on the basis of the data of the photoacoustic wave reception signals along the single scan line and the timing of starting a photoacoustic wave receiving operation, which is output from the sequence control unit 14. A specific insertion depth detection method performed by the insertion depth detection unit 9 will be described in detail below.

Upon detection of a reception signal for which the intensity of a reception signal related to the photoacoustic wave is greater than or equal to a certain value, the insertion depth detection unit 9 determines that a reception signal corresponding to the photoacoustic wave from the photoacoustic wave generation unit 21 has been detected, and can sense the insertion device 12.

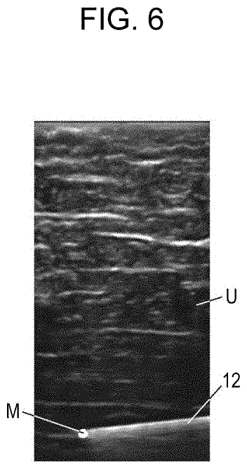

Further, the insertion depth detection unit 9 causes the display unit 8 to display the detected insertion depth of the insertion device 12 via the notification unit 11 and the display control unit 7. At this time, for example, as illustrated in FIG. 6, a marker M can be displayed at the position of the photoacoustic wave generation unit 21 of the insertion device 12 in an ultrasound image U displayed on the display unit 8. This makes it easy for the user to visually understand the insertion depth of the insertion device 12.

The depth setting unit 10 of the processor 19 sets a limit depth for the insertion depth of the insertion device 12. At this time, the depth setting unit 10 sets the depth input by the user through the operating unit 16 as the limit depth. The limit depth refers to the limit of depth beyond which advancement of the insertion device 12 is inhibited. For example, the distance from the body surface of the subject on which the probe 18 is placed to a position near an artery of the subject can be set as a limit depth.

The notification unit 11 of the processor 19 provides a notification to the user in a case where the insertion depth of the insertion device 12 detected by the insertion depth detection unit 9 is deeper than the limit depth set by the depth setting unit 10. For example, the notification unit 11 can provide a warning display on the display unit 8 via the display control unit 7, indicating that the insertion depth of the insertion device 12 is deeper than the limit depth. At this time, for example, the notification unit 11 can provide a warning display by displaying a text message and an image showing a warning on the display unit 8.

The apparatus control unit 15 of the processor 19 controls each unit of the ultrasound diagnostic apparatus 1A in accordance with a program stored in advance in the storage unit 17 or the like and the user's operation through the operating unit 16.

The display control unit 7 of the processor 19 performs predetermined processing on the ultrasound image generated by the ultrasound image generation unit 6, the insertion depth of the insertion device 12 detected by the insertion depth detection unit 9, and so on under control of the apparatus control unit 15 to generate an image that can be displayed on the display unit 8.

The display unit 8 of the ultrasound diagnostic apparatus 1A is configured to display an image generated by the display control unit 7 and includes, for example, a display device such as an LCD (Liquid Crystal Display).

The operating unit 16 of the ultrasound diagnostic apparatus 1A, through which the user performs an input operation, can be configured to include a keyboard, a mouse, a trackball, a touchpad, a touch panel, and the like.

The storage unit 17 is configured to store an operation program and the like of the ultrasound diagnostic apparatus 1A and can be implemented using a recording medium such as an HDD (Hard Disc Drive), an SSD (Solid State Drive), an FD (Flexible Disc), an MO disc (Magneto-Optical disc), an MT (Magnetic Tape), a RAM (Random Access Memory), a CD (Compact Disc), a DVD (Digital Versatile Disc), an SD card (Secure Digital card), or a USB memory (Universal Serial Bus memory), a server, or the like.

The processor 19 having the transmitting unit 3, the receiving unit 4, the data separation unit 5, the ultrasound image generation unit 6, the display control unit 7, the insertion depth detection unit 9, the depth setting unit 10, the notification unit 11, the sequence control unit 14, and the apparatus control unit 15 is constituted by a CPU (Central Processing Unit) and a control program for causing the CPU to perform various processing operations, or may be configured using a digital circuit. The transmitting unit 3, the receiving unit 4, the data separation unit 5, the ultrasound image generation unit 6, the display control unit 7, the insertion depth detection unit 9, the depth setting unit 10, the notification unit 11, the sequence control unit 14, and the apparatus control unit 15 may be configured to be partially or entirely integrated into a single CPU.

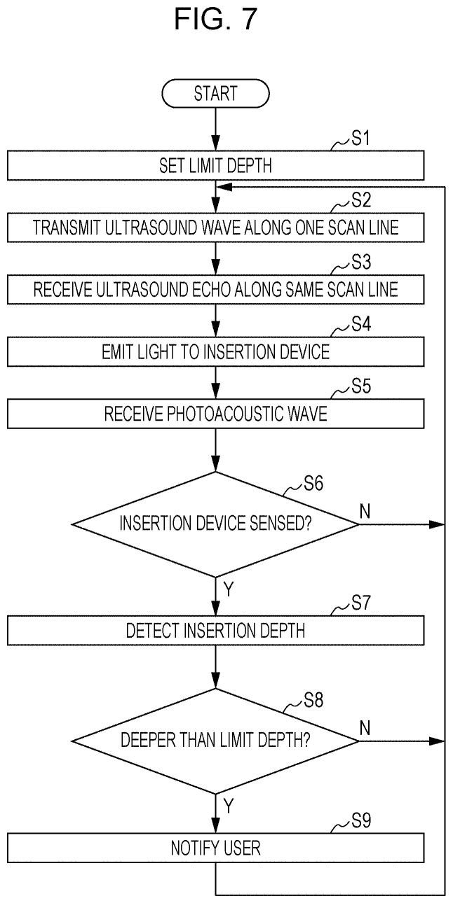

Next, the operation of the ultrasound diagnostic apparatus 1A according to Embodiment 1 will be described in detail with reference to a flowchart illustrated in FIG. 7.

First, in step S1, the depth setting unit 10 sets a depth input from the user through the operating unit 16 as a limit depth. At this time, for example, the user can input a typical distance from the body surface to an inhibition site through which advancement of the insertion device 12 is inhibited, such as an artery, as the depth to be set. Alternatively, for example, the user can input the depth to be set by checking a previously captured ultrasound image and the like of the subject. For example, the apparatus control unit 15 can accept a position designated by the user through the operating unit 16 in the ultrasound image displayed on the display unit 8. In this case, the user can designate a position in the ultrasound image displayed on the display unit 8 through the operating unit 16 to input the depth to be set.

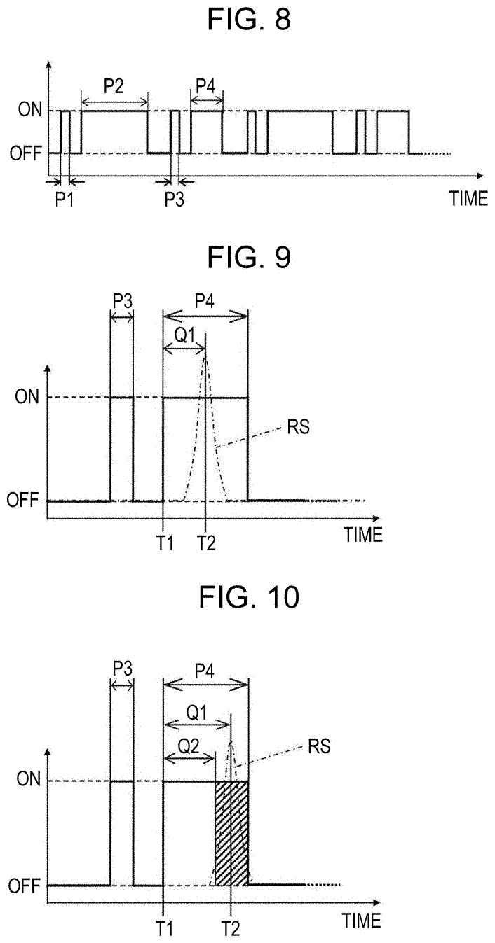

Then, in steps S2 to S5, as conceptually illustrated in FIG. 8, the sequence control unit 14 controls the timing of transmitting an ultrasound wave, the timing of receiving an ultrasound echo, the timing of emitting light from the light source 13, and the timing of receiving a photoacoustic wave.

First, in step S2, the sequence control unit 14 controls the transmitting unit 3 so that the transmission of an ultrasound wave to the subject along a single scan line is performed by the array transducer 2 during a period P1.

In a case where the transmission of an ultrasound wave to the subject is complete, in step S3, the sequence control unit 14 controls the receiving unit 4 so that an ultrasound echo receiving operation along the same scan line as the scan line of the ultrasound wave transmitted in step S2 is performed via the array transducer 2 during a period P2.

In a case where the ultrasound echo receiving operation in step S3 is complete, in step S4, the sequence control unit 14 controls the light source 13 so that the insertion device 12 is irradiated with light from the light source 13 during a period P3.

In a case where the irradiation of the insertion device 12 with light from the light source 13 is complete, in step S5, the sequence control unit 14 controls the receiving unit 4 so that a photoacoustic wave receiving operation along the same scan line as that of the ultrasound wave transmitted in step S2 is performed via the array transducer 2 during a period P4. In this manner, the sequence control unit 14 controls the transmitting unit 3, the receiving unit 4, and the light source 13 so that each time reception of an ultrasound echo along a single scan line is performed, reception of a photoacoustic wave along the same scan line is performed.

As used here, the time of receiving an ultrasound echo is the sum of the time taken until an ultrasound wave transmitted from the array transducer 2 reaches the subject's site being examined and the time taken until the reflected ultrasound echo reaches the array transducer 2, that is, the time taken for the ultrasound wave to reciprocate over a distance from the site being examined to the array transducer 2. On the other hand, the time of receiving a photoacoustic wave corresponds to the one way time taken for a photoacoustic wave generated by the photoacoustic wave generation unit 21 of the insertion device 12 to reach the array transducer 2. Thus, the period P4 during which a photoacoustic wave receiving operation is performed is half the period P2 during which an ultrasound echo receiving operation is performed.

Then, in step S6, the insertion depth detection unit 9 determines whether the insertion device 12 is sensed from the photoacoustic wave reception signal received in step S5, that is, whether the photoacoustic wave generated from the photoacoustic wave generation unit 21 of the insertion device 12 is detected. If it is determined in step S6 that the insertion device 12 is not sensed, the processing of steps S2 to S6 is performed for the next scan line. In this manner, the processing of steps S2 to S6 is sequentially performed for each scan line until the insertion device 12 is sensed by the insertion depth detection unit 9. If the insertion depth detection unit 9 determines in step S6 that the insertion device 12 is sensed, the process proceeds to step S7.

In step S7, the insertion depth detection unit 9 detects the insertion depth of the insertion device 12 on the basis of the timing of receiving a photoacoustic wave, which is output from the sequence control unit 14, and a photoacoustic wave detection signal. For example, as conceptually illustrated in FIG. 9, the insertion depth detection unit 9 multiplies a time interval Q1 from a time point T1 at which the array transducer 2 starts a photoacoustic wave receiving operation to a time point T2 at which a photoacoustic wave reception signal RS corresponding to the photoacoustic wave generated from the photoacoustic wave generation unit 21 of the insertion device 12 is detected, that is, the time point T2 at which the insertion device 12 is detected, by the sound velocity of the photoacoustic wave to obtain a distance from the body surface to the photoacoustic wave generation unit 21 of the insertion device 12, that is, the insertion depth of the insertion device 12.

In a case where the insertion depth of the insertion device 12 is detected in step S7, in step S8, the notification unit 11 determines whether the detected insertion depth of the insertion device 12 is deeper than the limit depth set in step S1. For example, as conceptually illustrated in FIG. 10, in a case where the time interval Q1 from the time point T1 at which the array transducer 2 starts a photoacoustic wave receiving operation to the time point T2 at which the insertion device 12 is sensed is larger than a time interval Q2 corresponding to the limit depth, the notification unit 11 determines that the insertion depth of the insertion device 12 is deeper than the limit depth. On the other hand, for example, although not illustrated, in a case where the time interval Q1 from the time point T1 at which the array transducer 2 starts a photoacoustic wave receiving operation to the time point T2 at which the insertion device 12 is sensed is less than or equal to the time interval Q2 corresponding to the limit depth, the notification unit 11 determines that the insertion depth of the insertion device 12 is less than or equal to the limit depth.

If the notification unit 11 determines that the insertion depth of the insertion device 12 is less than or equal to the limit depth, the processing of steps S2 to S7 is performed for the next scan line to detect the insertion depth of the insertion device 12, and the notification unit 11 determines whether the detected insertion depth is deeper than the limit depth. In this manner, as long as the insertion depth of the insertion device 12 detected in step S7 is less than or equal to the limit depth, the processing of steps S2 to S8 is repeatedly performed sequentially for each scan line.

If the notification unit 11 determines in step S8 that the insertion depth of the insertion device 12 is deeper than the limit depth, the process proceeds to step S9, and the notification unit 11 provides a notification to the user.

In a case where a notification is provided to the user in step S9, steps S2 to S9 are repeated until the insertion depth of the insertion device 12 detected in step S7 is less than or equal to the limit depth, and a notification is continuously provided to the user.

As described above, in the ultrasound diagnostic apparatus 1A according to Embodiment 1, each time reception of an ultrasound echo along a single scan line is performed, the insertion depth of the insertion device 12 is detected using a photoacoustic wave, and whether the detected insertion depth is deeper than the limit depth is determined. In addition, a notification is provided to the user in a case where the detected insertion depth is deeper than the limit depth. This enables rapid detection of the insertion depth of the insertion device 12 and enables the user to immediately take measures in a case where the insertion device 12 approaches an inhibition site through which advancement of the insertion device 12 is unwanted.

The insertion depth detection unit 9 detects a reception signal for which the intensity of a photoacoustic wave reception signal is greater than or equal to a certain value to sense the insertion device 12. In actuality, however, a photoacoustic wave reception signal received by the receiving unit 4 generally includes noise. The noise may cause erroneous sensing of the insertion device 12. Removing noise from the photoacoustic wave reception signal can prevent erroneous sensing of the insertion device 12. For example, specifically, in a case where the difference between the intensity of a reception signal corresponding to a photoacoustic wave from the photoacoustic wave generation unit 21 and the intensity of a reception signal corresponding to noise is greater than or equal to a certain value, for example, 20 dB, the insertion depth detection unit 9 can determine that the reception signal corresponding to the photoacoustic wave from the photoacoustic wave generation unit 21 is detected, and can sense the insertion device 12.

The notification unit 11 can provide a warning display on the display unit 8 via the display control unit 7, indicating that the insertion depth of the insertion device 12 is deeper than the limit depth. However, the present invention is not limited to this form. For example, although not illustrated, a notification can be provided to the user by freezing ultrasound images sequentially displayed on the display unit 8. For example, although not illustrated, in a case where the ultrasound diagnostic apparatus 1A is provided with an audio generation device, the notification unit 11 can provide a notification to the user by generating a warning sound such as voice. For example, although not illustrated, in a case where the probe 18 is provided with a device that generates vibration, the notification unit 11 can provide a notification to the user by slightly vibrating the probe 18. For example, although not illustrated, in a case where a warning light is attached to a housing or the like of the ultrasound diagnostic apparatus 1A, the notification unit 11 can provide a notification to the user by turning on, blinking, and the like of the warning light.

For example, the notification unit 11 can provide a notification to the user by removing or graying out the display of the marker M illustrated in FIG. 6 in a case where the insertion depth of the insertion device 12 is less than or equal to the limit depth, and by highlighting the display of the marker M on the display unit 8 in a case where the insertion depth of the insertion device 12 is deeper than the limit depth.

In addition, for example, the notification unit 11 can change the color of a tip portion of the insertion device 12 displayed on the display unit 8, that is, the color of the marker M, in accordance with the difference between the insertion depth of the insertion device 12 and the limit depth. At this time, for example, in a case where the difference between the insertion depth of the insertion device 12 and the limit depth is greater than or equal to 10 mm, the notification unit 11 can display the marker M in green. In a case where the difference between the insertion depth of the insertion device 12 and the limit depth is greater than 1 mm and less than 10 mm, the notification unit 11 can display the marker M in yellow. In a case where the difference between the insertion depth of the insertion device 12 and the limit depth is less than or equal to 1 mm, the notification unit 11 can display the marker M in red.

While the depth setting unit 10 sets the limit depth beyond which advancement of the insertion device 12 is inhibited, the depth setting unit 10 may set a target depth that is an intended depth finally reached by the insertion device 12. For example, the depth setting unit 10 can set, as target depths, the depth of a site to which the user desires to inject medication and the depth of a site from which the user desires to take a sample. In this case, for example, the notification unit 11 can provide a notification to the user in a case where the insertion device 12 has reached the target depth, instead of providing a notification to the user in a case where the insertion device 12 has reached the limit depth. Alternatively, the notification unit 11 can provide a notification to the user at the time point when the insertion device 12 reaches the target depth and at the time point when the insertion device 12 reaches the limit depth. In this case, for example, the notification unit 11 can provide a notification to the user at the time point when the insertion device 12 reaches the target depth and at the time point when the insertion device 12 reaches the limit depth by using different notification methods such as changing the color of the marker M to be displayed on the display unit 8.

### Embodiment 2

In Embodiment 1, each time transmission and reception of an ultrasound wave along a single scan line are performed, a photoacoustic wave is received along the same scan line as the scan line along which the ultrasound wave is transmitted and received. Alternatively, a photoacoustic wave may be received each time transmission and reception of ultrasound waves along a plurality of scan lines are performed.

An ultrasound diagnostic apparatus 1A according to Embodiment 2 has the same configuration as that of the ultrasound diagnostic apparatus 1A according to Embodiment 1.

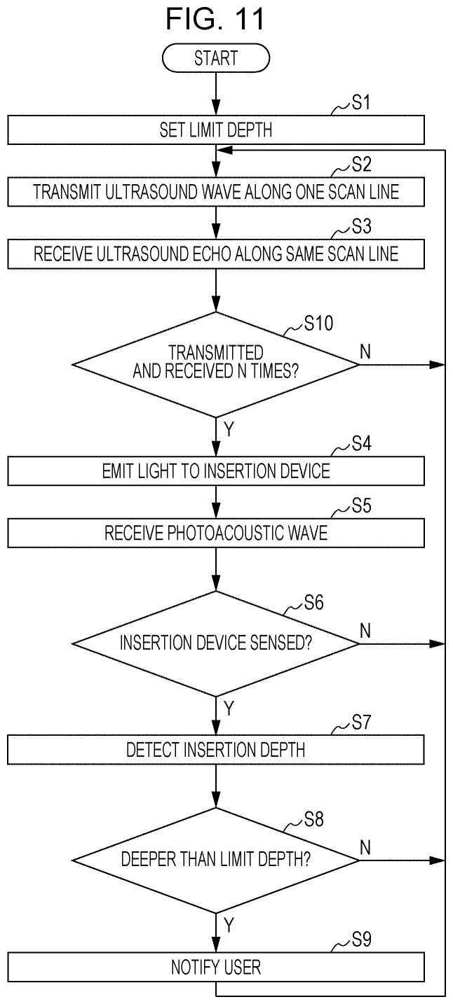

FIG. 11 is a flowchart illustrating the operation of the ultrasound diagnostic apparatus 1A according to Embodiment 2. In the flowchart according to Embodiment 2 illustrated in FIG. 11, step S10 is added immediately after step S3 in the flowchart according to Embodiment 1 illustrated in FIG. 7, and steps S1 to S3 and S4 to S9 according to Embodiment 2 are the same as those according to Embodiment 1.

In the operation of the ultrasound diagnostic apparatus 1A in Embodiment 2, first, in step S1, a limit depth for the insertion device 12 is set.

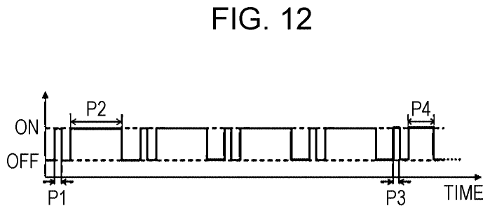

Then, in steps S2, S3, S10, S4, and S5, as conceptually illustrated in FIG. 12, the sequence control unit 14 controls the timing of transmitting an ultrasound wave, the timing of receiving an ultrasound echo, the timing of emitting light from the light source 13, and the timing of receiving a photoacoustic wave.

First, in step S2, the sequence control unit 14 controls the transmitting unit 3 so that the transmission of an ultrasound wave to the subject along a single scan line is performed by the array transducer 2 during a period P1. In a case where the transmission of an ultrasound wave in step S2 is complete, in step S3, the sequence control unit 14 controls the receiving unit 4 so that an ultrasound echo receiving operation along the same scan line as the scan line of the ultrasound wave transmitted in step S2 is performed via the array transducer 2 during a period P2.

Then, in step S10, the apparatus control unit 15 determines whether the transmission and reception of an ultrasound wave are consecutively performed N times by the array transducer 2. Here, N is a natural number greater than or equal to 2. If it is determined in step S10 that the transmission and reception of an ultrasound wave are not consecutively performed N times, the process returns to step S2, and an ultrasound wave is transmitted along the next single scan line. Then, in step S3, an ultrasound echo is received along the same scan line as the scan line along which the ultrasound wave is transmitted. In this manner, the processing of steps S2, S3, and S10 is repeatedly performed for a plurality of scan lines sequentially until it is determined that the transmission and reception of an ultrasound wave are consecutively performed N times.

If it is determined in step S10 that the transmission and reception of an ultrasound wave are consecutively performed N times, the process proceeds to step S4. In step S4, the sequence control unit 14 controls the light source 13 to emit light to the photoacoustic wave generation unit 21 of the insertion device 12.

Then, in step S5, the sequence control unit 14 controls the receiving unit 4 so that a photoacoustic wave receiving operation along the scan line for which the ultrasound echo receiving operation is finally performed in step S3 is performed via the array transducer 2.

In this manner, each time the transmission and reception of an ultrasound wave are performed N times, the light source 13 emits light and a photoacoustic wave receiving operation is performed via the array transducer 2. In the example illustrated in FIG. 12, N=4, and immediately after the transmission of an ultrasound wave and an ultrasound echo receiving operation are alternately performed four times, light is emitted from the light source 13 and a photoacoustic wave receiving operation is performed via the array transducer 2.

In a case where the photoacoustic wave receiving operation in step S5 is complete, in step S6, the insertion depth detection unit 9 determines whether the insertion device 12 is sensed. If it is determined in step S6 that the insertion device 12 is not sensed, the process returns to step S2, and the transmission of an ultrasound wave to the subject is performed. At this time, the array transducer 2 transmits an ultrasound wave along the scan line subsequent to the scan line along which an ultrasound wave is transmitted in step S2 of the previous round and for which the receiving operation is finally performed in step S3 of the previous round, that is, along the scan line subsequent to the scan line for which the photoacoustic wave receiving operation is performed in step S5. Then, in step S3, an ultrasound echo receiving operation is performed via the array transducer 2 along the scan line along which an ultrasound wave is transmitted in immediately preceding step S2.

In a case where the ultrasound echo receiving operation is performed via the array transducer 2 in step S3, in step S10, it is determined whether the transmission and reception of an ultrasound wave are consecutively performed N times. The processing of steps S2, S3, and S10 is repeatedly performed for a plurality of scan lines sequentially until it is determined in step S10 that the transmission and reception of an ultrasound wave are consecutively performed N times. If it is determined in step S10 that the transmission and reception of an ultrasound wave are consecutively performed N times, the process proceeds to step S4.

Then, in steps S4 and S5, light is emitted from the light source 13, and a photoacoustic wave receiving operation is performed via the array transducer 2. In step S6, the insertion depth detection unit 9 determines whether the insertion device 12 is sensed. In this manner, steps S2, S3, S10, and S4 to S6 are repeatedly performed until it is determined in step S6 that the insertion device 12 is sensed. If it is determined in step S6 that the insertion device 12 is sensed, the process proceeds to step S7.

Then, if the insertion depth detection unit 9 detects the insertion depth of the insertion device 12 in step S7, in step S8, the notification unit 11 determines whether the insertion depth of the insertion device 12 is deeper than a limit depth. If it is determined in step S8 that the insertion depth of the insertion device 12 is less than or equal to the limit depth, in step S2, the transmission of an ultrasound wave along the next scan line is performed, and in step S3, an ultrasound echo receiving operation along the same scan line is performed. If it is determined in step S8 that the insertion depth of the insertion device 12 is deeper than the limit depth, the process proceeds to step S9, and the notification unit 11 provides a notification to the user.

As described above, in the ultrasound diagnostic apparatus 1A of Embodiment 2 of the present invention, each time the transmission of ultrasound waves and the operation of receiving ultrasound echoes along a plurality of scan lines are performed, a photoacoustic wave receiving operation is performed. This can reduce the load imposed on the ultrasound diagnostic apparatus 1A for calculation compared with a case where each time a photoacoustic wave receiving operation is performed each time an ultrasound echo receiving operation along a single scan line is performed. In the ultrasound diagnostic apparatus 1A of Embodiment 2 of the present invention, furthermore, the number of times a photoacoustic wave receiving operation is performed is simply smaller than that in a case where a photoacoustic wave receiving operation is performed each time an ultrasound echo receiving operation along a single scan line is performed. This can reduce the time required to form an ultrasound image of one frame. For the reasons described above, the ultrasound diagnostic apparatus 1A of Embodiment 2 of the present invention can more rapidly generate an ultrasound image and sense the insertion device 12.

In Embodiment 2, each time an ultrasound echo receiving operation is performed along a plurality of scan lines, a photoacoustic wave receiving operation is performed along a single scan line. Alternatively, each time an ultrasound echo receiving operation is performed along a plurality of scan lines, a photoacoustic wave receiving operation may be performed along a plurality of scan lines. For example, although not illustrated, the emission of light from the light source 13 and a photoacoustic wave receiving operation via the array transducer 2 can be sequentially performed so as to perform, each time an ultrasound echo receiving operation is performed along M scan lines, a photoacoustic wave receiving operation along the M scan lines for which the ultrasound echo receiving operation is performed. This makes it possible to rapidly generate an ultrasound image and sense the insertion device 12 while maintaining the detection accuracy of the insertion device 12.

In Embodiment 2, furthermore, each time light is emitted from the light source 13 once, a photoacoustic wave receiving operation along a single scan line is performed via the array transducer 2. Alternatively, applying so-called parallel reception technology to the photoacoustic wave receiving operation can increase, in a virtual fashion, the number of scan lines for which a receiving operation is performed. Here, parallel reception technology refers to a technology generally known as ultrasound reception technology in which ultrasound echoes obtained by a single transmission of an ultrasound wave are received along a plurality of scan lines at the same time. With the application of parallel reception to the photoacoustic wave receiving operation, for example, parallel reception performed K times can increase, in a virtual fashion, the number of scan lines for a photoacoustic wave by K times while maintaining the time required to form an ultrasound image of one frame, resulting in improved accuracy of sensing of the insertion device 12 by the insertion depth detection unit 9.

In step S10 in Embodiment 2, the apparatus control unit 15 determines whether the transmission and reception of an ultrasound wave are consecutively performed N times. The number N may be recorded in advance in the storage unit 17 or the like and read by the apparatus control unit 15 each time it is required, or may be set by the user through the operating unit 16.

### Embodiment 3

In Embodiments 1 and 2, the user inputs a limit depth in advance through the operating unit 16, and the depth setting unit 10 sets a limit depth. Alternatively, a pre-scan may be performed to acquire an ultrasound image before the insertion device 12 is sensed, and a limit depth may be set using image analysis.

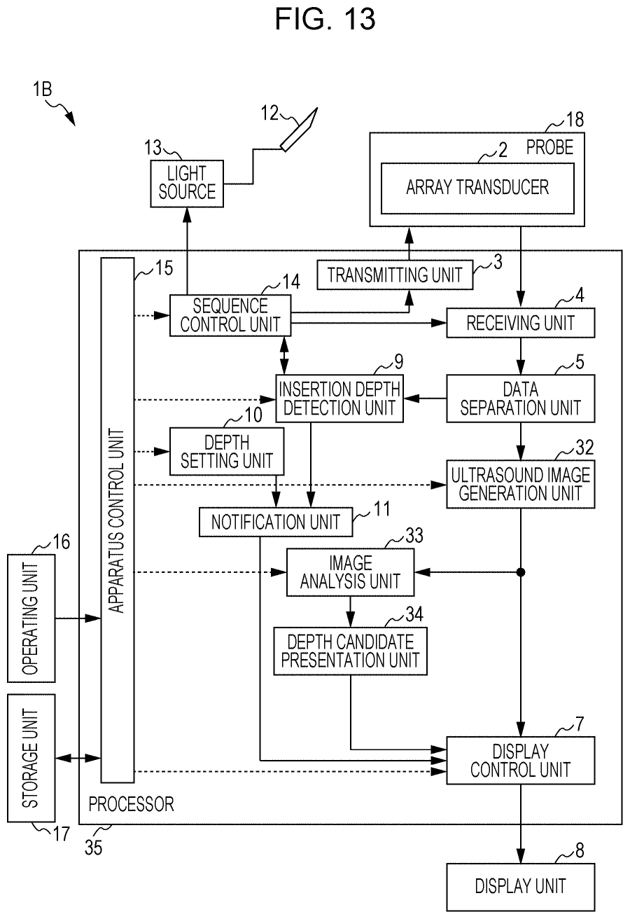

FIG. 13 illustrates a configuration of an ultrasound diagnostic apparatus 1B of Embodiment 3. The ultrasound diagnostic apparatus 1B illustrated in FIG. 13 has the same configuration as that of the ultrasound diagnostic apparatus 1A of Embodiment 1 illustrated in FIG. 1, except that the ultrasound diagnostic apparatus 1B includes an ultrasound image generation unit 32 in place of the ultrasound image generation unit 6 and further includes an image analysis unit 33 and a depth candidate presentation unit 34.

In the ultrasound diagnostic apparatus 1B of Embodiment 3, the ultrasound image generation unit 32 is connected to the data separation unit 5, and the display control unit 7 and the image analysis unit 33 are connected to the ultrasound image generation unit 32. Further, the depth candidate presentation unit 34 is connected to the image analysis unit 33, and the display control unit 7 is connected to the depth candidate presentation unit 34. Further, the ultrasound image generation unit 32 and the image analysis unit 33 are connected to the apparatus control unit 15.

The transmitting unit 3, the receiving unit 4, the data separation unit 5, the display control unit 7, the insertion depth detection unit 9, the depth setting unit 10, the notification unit 11, the sequence control unit 14, the apparatus control unit 15, the ultrasound image generation unit 32, the image analysis unit 33, and the depth candidate presentation unit 34 constitute a processor 35.

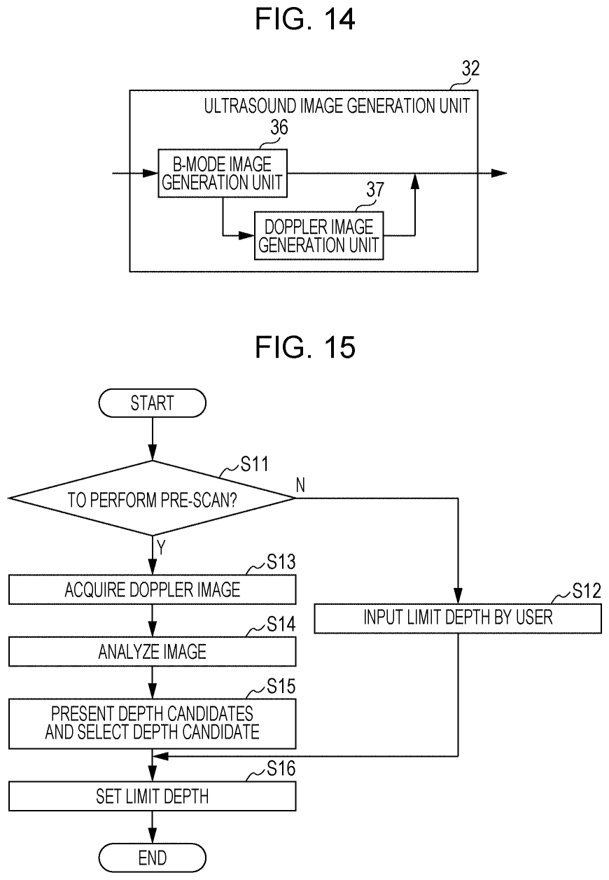

FIG. 14 illustrates an internal configuration of the ultrasound image generation unit 32 of the processor 35. The ultrasound image generation unit 32 has a B-mode image generation unit 36 and a Doppler image generation unit 37, and the Doppler image generation unit 37 is connected to the B-mode image generation unit 36.

Here, the B-mode image generation unit 36 has the same configuration as the ultrasound image generation unit 6 in Embodiment 1 illustrated in FIGS. 1 and 5, and, although not illustrated, has the signal processing unit 29, the DSC 30, and the image processing unit 31.

The Doppler image generation unit 37 of the ultrasound image generation unit 32 generates a Doppler image using, for example, a color Doppler method. Although not illustrated, the Doppler image generation unit 37 performs frequency analysis of an ultrasound reception signal to calculate a Doppler shift frequency, and acquires, as Doppler data, information on the relative speed of movement of tissue of the subject with respect to the array transducer 2. Further, the Doppler image generation unit 37 converts each piece of Doppler data in each piece of tissue into color information corresponding to the speed thereof and performs various necessary image processing operations, such as gradation processing, to generate a color Doppler image signal, that is, a Doppler image. The generated Doppler image is combined with, for example, an ultrasound image generated by the B-mode image generation unit 36 so as to be superimposed on the corresponding piece of tissue in the ultrasound image.

The image analysis unit 33 of the processor 35 performs image analysis on the ultrasound image generated by the ultrasound image generation unit 32 to detect an inhibition site that is a site through which advancement of the insertion device 12 is inhibited. For example, the image analysis unit 33 can detect blood flow using the Doppler image generated by the ultrasound image generation unit 32 to detect blood vessels such as a vein and an artery as inhibition sites.

The depth candidate presentation unit 34 of the processor 35 presents a plurality of depth candidates related to the limit depth to the user on the basis of the inhibition site detected by the image analysis unit 33. For example, in a case where a plurality of inhibition sites are detected, the depth candidate presentation unit 34 can determine, as depth candidates, the depth at the shallowest position and the depth at the deepest position within an area occupied by each inhibition site and display these depth candidates on the display unit 8 via the display control unit 7.

Next, a limit depth setting operation in Embodiment 3 will be described with reference to a flowchart illustrated in FIG. 15.

First, in step S11, the apparatus control unit 15 determines whether to perform a pre-scan. At this time, for example, the apparatus control unit 15 can determine whether to perform a pre-scan in accordance with an instruction given by the user through the operating unit 16. At this time, for example, the apparatus control unit 15 displays a message asking whether to perform a pre-scan on the display unit 8. In a case where the user refers to the message and inputs an instruction to perform a pre-scan or an instruction not to perform a pre-scan through the operating unit 16, the apparatus control unit 15 determines whether to perform a pre-scan in accordance with the instruction given by the user. In a case where no pre-scan is to be performed, as in Embodiments 1 and 2, in step S12, the user inputs a limit depth, and in step S16, the depth setting unit 10 sets a limit depth.

If it is determined in step S11 that a pre-scan is to be performed, the process proceeds to step S13, and the ultrasound image generation unit 32 generates an ultrasound image with which the Doppler image is combined.

In a case where an ultrasound image is generated in step S13, in step S14, the image analysis unit 33 performs image analysis on the Doppler image generated in step S13 to detect blood flow to detect an inhibition site in the ultrasound image.

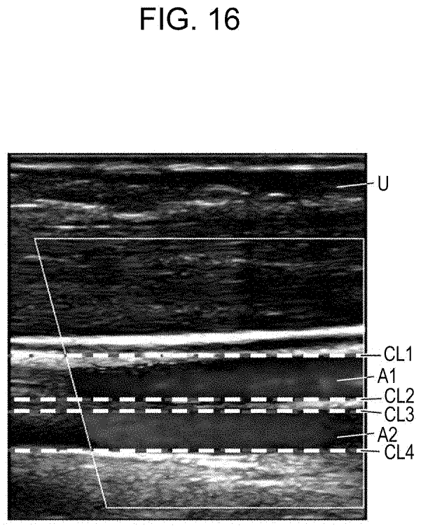

In a case where an inhibition site is detected in step S14, the depth candidate presentation unit 34 presents a plurality of depth candidates related to the limit depth to the user on the basis of the inhibition site. For example, as illustrated in FIG. 16, the depth candidate presentation unit 34 can superimpose and display a candidate line CL1 indicating the shallowest position within an area occupied by an inhibition site A1, a candidate line CL2 indicating the deepest position within the area occupied by the inhibition site A1, a candidate line CL3 indicating the shallowest position within an area occupied by an inhibition site A2, and a candidate line CL4 indicating the deepest position within the area occupied by the inhibition site A2 on the ultrasound image U to present a plurality of depth candidates related to the limit depth to the user.

The user can select one of the plurality of depth candidates presented by the depth candidate presentation unit 34. In this manner, in a case where one depth candidate is selected, in step S16, the depth setting unit 10 sets a limit depth from the depth candidate selected by the user, and the limit depth setting operation in Embodiment 3 is finished.

In a case where the limit depth setting operation according to the flowchart illustrated in FIG. 15 is complete, the insertion device 12 is sensed. At this time, for example, in accordance with steps S2 to S9 in Embodiment 1 illustrated in FIG. 7, the ultrasound diagnostic apparatus 1B performs transmission of an ultrasound wave, an ultrasound echo receiving operation, light emission, and a photoacoustic wave receiving operation, senses the insertion device 12, and provides a notification to the user in a case where the insertion depth of the insertion device 12 is deeper than the limit depth.

As described above, the ultrasound diagnostic apparatus 1B of Embodiment 3 can perform a pre-scan to acquire a Doppler image, analyze the Doppler image to detect blood flow, and present a plurality of depth candidates related to the limit depth to the user. This can reduce the load imposed on the user to determine a limit depth.

In Embodiment 3, the depth setting unit 10 sets a limit depth from a depth candidate selected by the user from among a plurality of depth candidates presented by the depth candidate presentation unit 34. Alternatively, a depth input by the user referring to the plurality of depth candidates through the operating unit 16 can be set as a limit depth. This enables the depth setting unit 10 to set a limit depth on the basis of, for example, a position determined by the user to be more suitable.

### Embodiment 4

In Embodiment 3, the image analysis unit 33 analyzes a Doppler image to detect an inhibition site. Alternatively, the image analysis unit 33 may perform image analysis on an ultrasound image formed of a B-mode image signal, called a B-mode image, to detect an inhibition site.

An ultrasound diagnostic apparatus 1B of Embodiment 4 has the same configuration as the ultrasound diagnostic apparatus 1B of Embodiment 3 illustrated in FIG. 13.

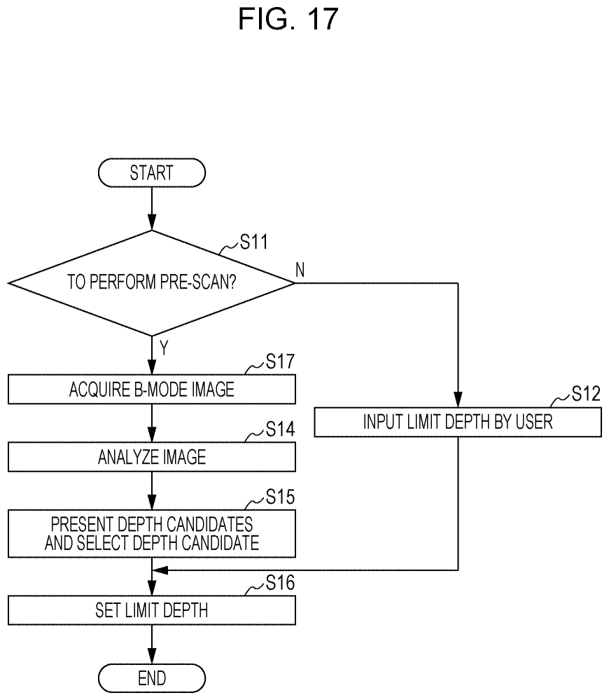

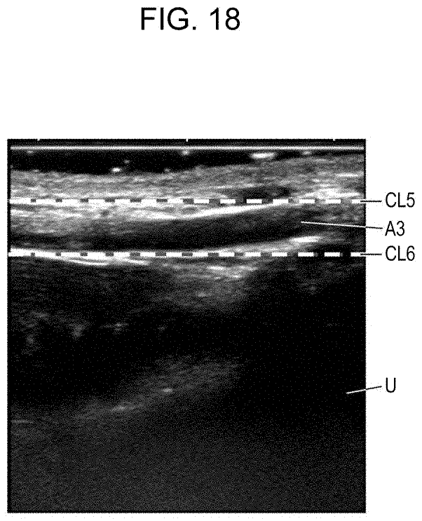

FIG. 17 depicts a flowchart illustrating a limit depth setting operation performed by the ultrasound diagnostic apparatus 1B in Embodiment 4. The flowchart illustrated in FIG. 17 is the same as the flowchart illustrated in FIG. 15, except that step S13 in Embodiment 3 illustrated in FIG. 15 is replaced by step S17.

First, in step S11, the apparatus control unit 15 determines whether to perform a pre-scan. If it is determined that no pre-scan is to be performed, in step S12, the user inputs a limit depth through the operating unit 16, and in step S16, a limit depth is set from the depth input by the user. If it is determined in step S11 that a pre-scan is to be performed, the process proceeds to step S17, and the ultrasound image generation unit 32 generates a B-mode image.

Then, in step S14, the image analysis unit 33 performs image analysis on the ultrasound image generated in step S17 to detect an inhibition site in the ultrasound image. For example, as illustrated in FIG. 18, the image analysis unit 33 detects a low-luminance continuous area in an ultrasound image as an inhibition site A3.

In a case where an inhibition site is detected in step S14, the depth candidate presentation unit 34 presents a plurality of depth candidates related to the limit depth to the user on the basis of the inhibition site. For example, as illustrated in FIG. 18, the depth candidate presentation unit 34 can superimpose and display a candidate line CL5 indicating the shallowest position and a candidate line CL6 indicating the deepest position within an area occupied by the inhibition site A3 on the ultrasound image U to present a plurality of depth candidates related to the limit depth to the user.

The user can select one of the plurality of depth candidates presented by the depth candidate presentation unit 34.

In a case where a depth candidate is selected by the user, in step S16, the depth setting unit 10 sets a limit depth from the depth candidate selected by the user, and the limit depth setting operation in Embodiment 4 is finished.

In a case where the limit depth setting operation according to the flowchart illustrated in FIG. 17 is complete, the insertion device 12 is sensed. At this time, for example, in accordance with steps S2 to S9 in Embodiment 1 illustrated in FIG. 7, the ultrasound diagnostic apparatus 1B performs transmission of an ultrasound wave, an ultrasound echo receiving operation, light emission, and a photoacoustic wave receiving operation, senses the insertion device 12, and provides a notification to the user in a case where the insertion depth of the insertion device 12 is deeper than the limit depth.

As described above, the ultrasound diagnostic apparatus 1B of Embodiment 4 can perform a pre-scan to acquire a B-mode image, perform image analysis on this ultrasound image to detect an inhibition site, and present a plurality of depth candidates related to the limit depth to the user. This can reduce the load imposed on the user to determine a limit depth.

The ultrasound diagnostic apparatus 1B of Embodiment 4 has the same configuration as that of the ultrasound diagnostic apparatus 1B of Embodiment 3. In Embodiment 4, however, since no Doppler image is generated, the ultrasound diagnostic apparatus 1B of Embodiment 4 may include the ultrasound image generation unit 6 in Embodiment 1 illustrated in FIGS. 1 and 5, in place of the ultrasound image generation unit 32 having the Doppler image generation unit 37.

In addition, image analysis is performed on a Doppler image to detect an inhibition site in Embodiment 3, whereas, in Embodiment 4, image analysis is performed on a B-mode image to detect an inhibition site. However, the ultrasound diagnostic apparatus 1B can also perform a pre-scan in accordance with an operation that allows selection of whether to perform image analysis on a Doppler image or perform image analysis on a B-mode image.

### Embodiment 5

In Embodiments 3 and 4, a plurality of depth candidates related to the limit depth are presented to the user on the basis of a result of image analysis performed on an ultrasound image. Alternatively, a limit depth may be automatically set on the basis of a result of image analysis.

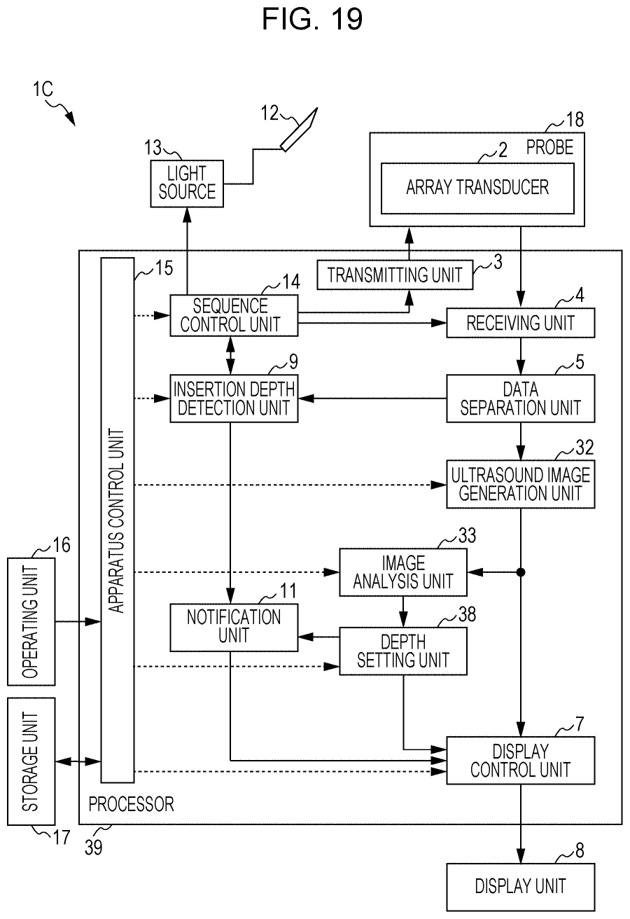

FIG. 19 illustrates a configuration of an ultrasound diagnostic apparatus 1C of Embodiment 5. In the ultrasound diagnostic apparatus 1C of Embodiment 5, a depth setting unit 38 is connected to the image analysis unit 33, and the depth setting unit 38 is connected to the display control unit 7, the notification unit 11, and the apparatus control unit 15. The ultrasound diagnostic apparatus 1C of Embodiment 5 has the same configuration as the ultrasound diagnostic apparatus 1B of Embodiment 3 illustrated in FIG. 13, except that the ultrasound diagnostic apparatus 1C does not include the depth candidate presentation unit 34 and that the depth setting unit 38 is connected to the image analysis unit 33.

The transmitting unit 3, the receiving unit 4, the data separation unit 5, the display control unit 7, the insertion depth detection unit 9, the notification unit 11, the sequence control unit 14, the apparatus control unit 15, the ultrasound image generation unit 32, the image analysis unit 33, and the depth setting unit 38 constitute a processor 39.

The depth setting unit 38 of the processor 39 is capable of automatically setting a limit depth on the basis of an inhibition site detected by the image analysis unit 33 performing image analysis on an ultrasound image and presenting the set limit depth to the user.

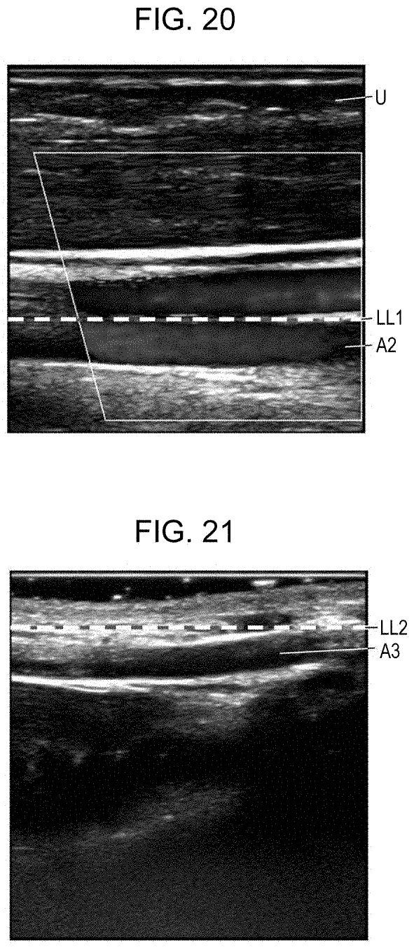

For example, in a case where the ultrasound image generation unit 32 generates a Doppler image, the image analysis unit 33 performs image analysis on the Doppler image to detect blood flow, and detects an inhibition site. At this time, the image analysis unit 33 can determine that the detected blood flow is blood flow in an artery or blood flow in a vein from the speed of the blood flow and time changes in the speed of the blood flow, and detect an artery as an inhibition site. In this case, for example, as illustrated in FIG. 20, the depth setting unit 38 can set the depth at the shallowest position within an area occupied by an inhibition site A2 as a limit depth. At this time, as illustrated in FIG. 20, the depth setting unit 38 can superimpose a depth setting line LL1 representing the limit depth on an ultrasound image U to be displayed on the display unit 8.

For example, in a case where the ultrasound image generation unit 32 generates a B-mode image, the image analysis unit 33 performs image analysis on the ultrasound image to detect an inhibition site. At this time, the image analysis unit 33 can detect a low-luminance continuous area as an area corresponding to an inhibition site. In this case, for example, as illustrated in FIG. 21, the depth setting unit 38 can set the depth at the shallowest position within an area occupied by an inhibition site A3 as a limit depth. At this time, as illustrated in FIG. 21, the depth setting unit 38 can superimpose a depth setting line LL2 representing the limit depth on an ultrasound image to be displayed on the display unit 8.

As in Embodiments 3 and 4, also in Embodiment 5, in a case where the limit depth setting operation is complete, the insertion device 12 is sensed. At this time, for example, in accordance with steps S2 to S9 in Embodiment 1 illustrated in FIG. 7, the ultrasound diagnostic apparatus 1C performs transmission of an ultrasound wave, an ultrasound echo receiving operation, light emission, and a photoacoustic wave receiving operation, senses the insertion device 12, and provides a notification to the user in a case where the insertion depth of the insertion device 12 is deeper than the limit depth.

### Embodiment 6

In Embodiments 1 to 5, a depth set before the insertion device 12 is sensed is constantly used as a limit depth. The actual limit depth may change with time depending on the angle at which the probe 18 touches the body surface of the subject, the strength with which the probe 18 is pressed against the subject, the heart beat rate, or the like. To address such movements of organs, the limit depth can be updated while the insertion device 12 is sensed.

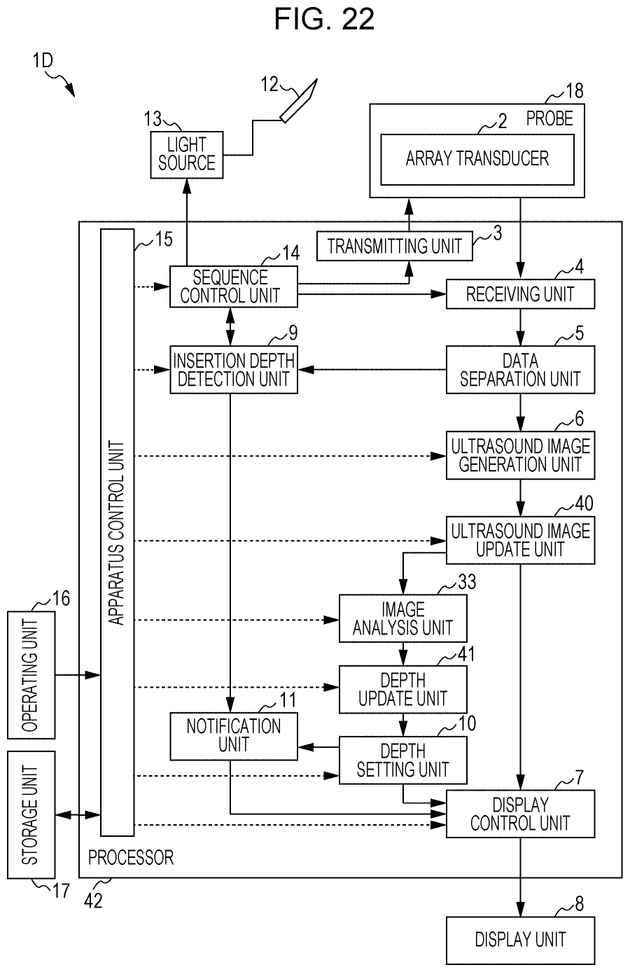

FIG. 22 illustrates a configuration of an ultrasound diagnostic apparatus 1D of Embodiment 6. The ultrasound diagnostic apparatus 1D of Embodiment 6 illustrated in FIG. 22 has the same configuration as the ultrasound diagnostic apparatus 1A of Embodiment 1 illustrated in FIG. 1, except that the ultrasound diagnostic apparatus 1D further includes an image analysis unit 33, an ultrasound image update unit 40, and a depth update unit 41 and that the depth setting unit 10 is connected to the depth update unit 41. The image analysis unit 33 in Embodiment 6 is the same as the image analysis unit 33 in Embodiment 3 illustrated in FIG. 13.

In the ultrasound diagnostic apparatus 1D of Embodiment 6, the ultrasound image update unit 40 is connected to the ultrasound image generation unit 6, and the display control unit 7 and the image analysis unit 33 are connected to the ultrasound image update unit 40. Further, the image analysis unit 33, the depth update unit 41, and the depth setting unit 10 are sequentially connected to the ultrasound image update unit 40, and the display control unit 7 and the notification unit 11 are connected to the depth setting unit 10. Further, the apparatus control unit 15 is connected to the ultrasound image update unit 40, the depth update unit 41, and the depth setting unit 10.

The transmitting unit 3, the receiving unit 4, the data separation unit 5, the ultrasound image generation unit 6, the display control unit 7, the insertion depth detection unit 9, the notification unit 11, the sequence control unit 14, the apparatus control unit 15, the image analysis unit 33, the ultrasound image update unit 40, the depth update unit 41, and the depth setting unit 10 constitute a processor 42.

Each time an ultrasound echo along a new scan line is received by the array transducer 2, the ultrasound image update unit 40 of the processor 42 updates an ultrasound image previously generated by the ultrasound image generation unit 6 by using a new ultrasound reception signal output from the array transducer 2.

Each time an ultrasound image is updated by the ultrasound image update unit 40, the depth update unit 41 of the processor 42 updates the limit depth on the basis of an area occupied by an inhibition site detected by the image analysis unit 33. The limit depth updated by the depth update unit 41 is output to the display control unit 7 and the notification unit 11 via the depth setting unit 10.

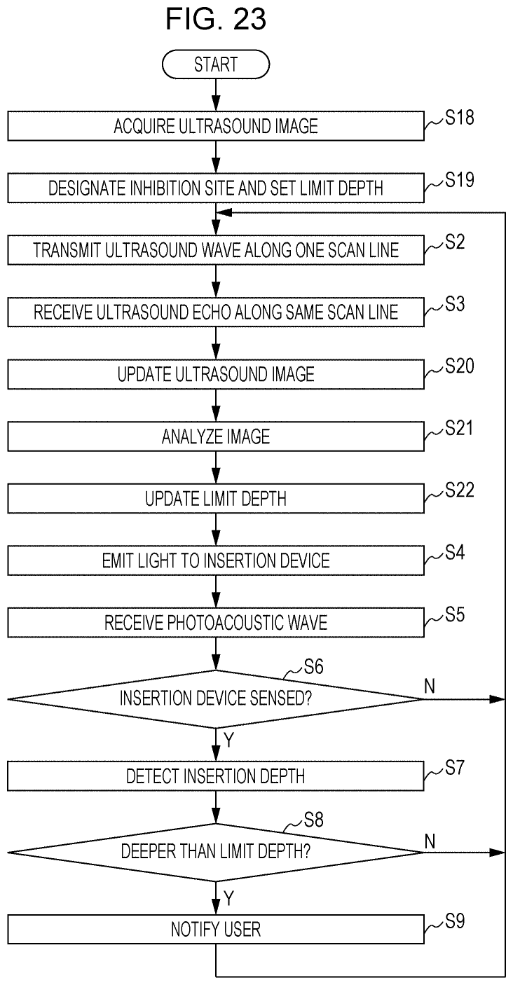

Next, the operation of the ultrasound diagnostic apparatus 1D in Embodiment 6 will be described with reference to a flowchart illustrated in FIG. 23. In the flowchart illustrated in FIG. 23, steps S2, S3, and S4 to S9 are the same as steps S2, S3, and S4 to S9 in Embodiment 1 illustrated in FIG. 7, respectively.

First, in step S18, the sequence control unit 14 controls the transmitting unit 3 and the receiving unit 4 to transmit and receive an ultrasound wave from the array transducer 2 to the subject. Further, an ultrasound reception signal output from the array transducer 2 upon receipt of an ultrasound echo is subjected to predetermined processing by the receiving unit 4 and the ultrasound image generation unit 6 to acquire an ultrasound image.

In a case where an ultrasound image is acquired in step S18, in step S19, the user designates an inhibition site in the ultrasound image acquired in step S18 by using the operating unit 16. In a case where a limit depth is input from the user through the operating unit 16, the depth setting unit 10 sets the depth input from the user as a limit depth.

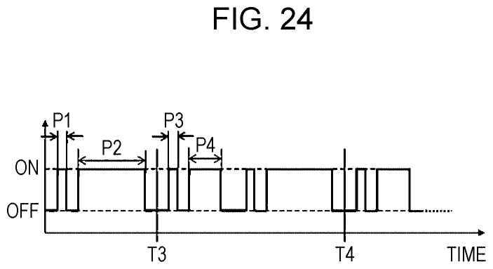

Then, in step S2, the sequence control unit 14 controls the transmitting unit 3 so that, as conceptually illustrated in FIG. 24, the transmission of an ultrasound wave to the subject along a single scan line is performed by the array transducer 2 during a period P1. In a case where the transmission of an ultrasound wave in step S2 is complete, in step S3, the sequence control unit 14 controls the receiving unit 4 so that an ultrasound echo receiving operation along the same scan line as the scan line of the ultrasound wave transmitted in step S2 is performed via the array transducer 2 during a period P2.

In a case where the ultrasound echo receiving operation in step S3 is complete, the process proceeds to step S20, and the ultrasound image update unit 40 updates the ultrasound image using the ultrasound reception signal obtained in step S3. For example, as conceptually illustrated in FIG. 24, the ultrasound image update unit 40 updates the ultrasound image at a time point T3 immediately after the ultrasound echo receiving operation is completed. At this time, the ultrasound image update unit 40 updates the ultrasound image by, for example, replacing the ultrasound reception signal that corresponds to the same scan line as the scan line for which the ultrasound echo receiving operation is performed in step S3 and that is used to generate an ultrasound image of the immediately preceding frame with a newly obtained ultrasound reception signal.

In a case where the update of the ultrasound image in step S20 is complete, in step S21, the image analysis unit 33 performs image analysis on the updated ultrasound image to detect the inhibition site designated by the user through the operating unit 16 in step S19. At this time, the image analysis unit 33 can detect the position designated by the user in step S19 by using, for example, a known technique such as so-called template matching, optical flow analysis, and feature point matching.

In a case where the image analysis in step S21 is complete, in step S22, the depth update unit 41 calculates and updates a limit depth for the updated ultrasound image from an area occupied by the inhibition site in the ultrasound image updated in step S20 and from the limit depth input from the user in step S19. At this time, the depth update unit 41 can calculate a limit depth by, for example, calculating a difference between the depth at the shallowest position within an area occupied by the inhibition site designated by the user in step S19 in the ultrasound image acquired in step S18 and the position of the depth input by the user in step S19 and subtracting the calculated difference from the depth at the shallowest position within an area occupied by the inhibition site in the ultrasound image updated in step S20.

Then, in step S4, as conceptually illustrated in FIG. 24, the sequence control unit 14 controls the light source 13 so that the insertion device 12 is irradiated with light from the light source 13 during a period P3. In a case where the irradiation with light in step S4 is complete, in step S5, the sequence control unit 14 controls the receiving unit 4 so that a photoacoustic wave receiving operation along the same scan line as that of the ultrasound wave transmitted in step S2 is performed via the array transducer 2 during a period P4.

Then, in step S6, the insertion depth detection unit 9 determines whether the insertion device 12 is sensed. If it is determined in step S6 that the insertion device 12 is not sensed, the process returns to step S2, and an ultrasound wave is transmitted to the subject from the array transducer 2 along the next scan line. Then, in step S3, an ultrasound echo receiving operation along the same scan line is performed.

In a case where the ultrasound echo receiving operation in step S3 is complete, in step S20, the ultrasound image is updated using a new ultrasound reception signal obtained through the receiving operation. At this time, for example, as illustrated in FIG. 24, after the ultrasound image is updated at the time point T3, the ultrasound image update unit 40 further updates the ultrasound image again at a time point T4 immediately after single transmission and reception of an ultrasound wave are completed. In this manner, the ultrasound image update unit 40 updates an ultrasound image each time an ultrasound echo is received along a new single scan line.

In a case where the ultrasound image is updated in step S20, in step S21, image analysis is performed on the updated ultrasound image, and in step S22, the limit depth is updated. Then, in a case where the emission of light in step S4 and the photoacoustic wave receiving operation in step S5 are complete, in step S6, it is determined whether the insertion device 12 is sensed.

If it is determined in step S6 that the insertion device 12 is sensed, the process proceeds to step S7, and the insertion depth of the insertion device 12 is detected from the photoacoustic wave reception signal obtained in step S5.

In a case where the insertion depth of the insertion device 12 is detected in step S7, in step S8, the notification unit 11 determines whether the detected depth is deeper than the limit depth finally updated in step S22. If it is determined that the depth detected in step S7 is less than or equal to the limit depth, the process returns to step S2. If it is determined that the depth detected in step S7 is deeper than the limit depth, the process proceeds to step S9, and the notification unit 11 provides a notification to the user.

As described above, in the ultrasound diagnostic apparatus 1D of Embodiment 6, each time an ultrasound echo is received along a new scan line, the ultrasound image is updated and further the limit depth is updated. Thus, even if the position of the actual limit depth changes with time depending on the angle at which the probe 18 touches the body surface of the subject, the strength with which the probe 18 is pressed against the subject, the heart beat rate, or the like, the user is able to immediately take measures in a case where the insertion device 12 approaches a site through which advancement of the insertion device 12 is unwanted.

In the operation of the ultrasound diagnostic apparatus 1D in Embodiment 6, an ultrasound image is acquired in step S18, and the user designates a position corresponding to a limit depth through the operating unit 16 in step S19, thereby setting a limit depth. Alternatively, the processing performed in steps S18 and S19 may be replaced with the limit depth setting operation described in Embodiments 3 to 5.

In a case where the processing performed in steps S18 and S19 in Embodiment 6 is replaced with the limit depth setting operation in Embodiments 3 and 4, for example, although not illustrated, the ultrasound diagnostic apparatus 1D of Embodiment 6 is provided with the depth candidate presentation unit 34, and the depth candidate presentation unit 34 presents a plurality of depth candidates to the user on the basis of an ultrasound image generated by the ultrasound image generation unit 6. Further, the user selects one of the plurality of depth candidates through the operating unit 16 to set a limit depth.

In a case where the processing performed in steps S18 and S19 in Embodiment 6 is replaced with the limit depth setting operation in Embodiment 5, for example, a limit depth is automatically set on the basis of an ultrasound image generated by the ultrasound image generation unit 6.

In addition, in Embodiment 6, the ultrasound image update unit 40 updates an ultrasound image each time an ultrasound echo is received along a new single scan line by the array transducer 2. Alternatively, an ultrasound image may be updated each time ultrasound echoes are received along a plurality of scan lines by the array transducer 2.

Accordingly, the ultrasound image update unit 40 less frequently updates an ultrasound image. This can reduce the computation load of the ultrasound diagnostic apparatus 1D and enables more rapid sensing of the insertion device 12.

From the foregoing description, an ultrasound diagnostic apparatus as set forth in Supplementary Note 1 below can be provided.

### [Supplementary Note 1]

An ultrasound diagnostic apparatus including:

a probe having an array transducer;

a transmission processor that transmits respective ultrasound beams from the array transducer to a subject along a plurality of scan lines;

an ultrasound image generation processor that converts ultrasound reception signals obtained from the array transducer in response to receipt of ultrasound echoes from the subject into an image to generate an ultrasound image of the subject;

an insertion device insertable into the subject and having a photoacoustic wave generation processor;

a light source that irradiates the photoacoustic wave generation processor of the insertion device with light to generate a photoacoustic wave from the photoacoustic wave generation processor;

a sequence control processor that controls the transmission processor and the light source so that the photoacoustic wave is received by the array transducer each time the array transducer receives an ultrasound echo along a predetermined number of scan lines;

an insertion depth detection processor that detects an insertion depth of the insertion device on the basis of a photoacoustic wave reception signal obtained by the array transducer; and

a notification processor that provides a notification to a user in a case where the insertion depth of the insertion device detected by the insertion depth detection processor is deeper than a determined depth.

### REFERENCE SIGNS LIST

1A, 1B, 1C, 1D ultrasound diagnostic apparatus
2 array transducer
3 transmitting unit
4 receiving unit
5 data separation unit
6, 32 ultrasound image generation unit
7 display control unit
8 display unit
9 insertion depth detection unit
10, 38 depth setting unit
11 notification unit
12 insertion device
13 light source
14 sequence control unit
15 apparatus control unit
16 operating unit
17 storage unit
18 probe
19, 35, 39, 42 processor
20 light guide member
21 photoacoustic wave generation unit
22 laser rod
23 flash lamp
24, 25 mirror
26 Q switch
27 amplifier unit
28 AD conversion unit
29 signal processing unit
30 DSC
31 image processing unit
33 image analysis unit
34 depth candidate presentation unit
36 B-mode image generation unit
37 Doppler image generation unit
40 ultrasound image update unit
41 depth update unit
A1, A2, A3 inhibition site
CL1, CL2, CL3, CL4, CL5, CL6 candidate line
E, FE tip portion
LL1, LL2 depth setting line
M marker
P1, P2, P3, P4 period
Q1, Q2 time interval
RS photoacoustic wave reception signal
T1, T2, T3, T4 time point
U ultrasound image

What is claimed is:

1. An ultrasound diagnostic apparatus comprising:
a probe having an array transducer;
an insertion device insertable into a subject and having a photoacoustic wave generation unit;
a light source that irradiates the photoacoustic wave generation unit of the insertion device with light to generate a photoacoustic wave from the photoacoustic wave generation unit; and
a processor,
wherein the processor is configured to
transmit respective ultrasound beams from the array transducer to the subject along a plurality of scan lines,
convert ultrasound reception signals obtained from the array transducer in response to receipt of ultrasound echoes from the subject into an image to generate an ultrasound image of the subject,
control transmission of the ultrasound beams and the light source so that the photoacoustic wave is received by the array transducer each time the array transducer receives an ultrasound echo along a predetermined number of scan lines,
detect an insertion depth of the insertion device on the basis of a photoacoustic wave reception signal obtained by the array transducer, and
provide a notification to a user in a case where the insertion depth of the insertion device is deeper than a determined depth.

2. The ultrasound diagnostic apparatus according to claim 1, wherein the processor is configured to control the transmission of the ultrasound beams and the light source so that the photoacoustic wave is received by the array transducer each time reception of an ultrasound echo along a single scan of the plurality of scan lines is performed.

3. The ultrasound diagnostic apparatus according to claim 1, wherein the processor is configured to control the transmission of the ultrasound beams and the light source so that the photoacoustic wave is received by the array transducer each time reception of ultrasound echoes along the plurality of scan lines is performed.

4. The ultrasound diagnostic apparatus according to claim 1, wherein the processor is configured to set the determined depth.

5. The ultrasound diagnostic apparatus according to claim 2, wherein the processor is configured to set the determined depth.

6. The ultrasound diagnostic apparatus according to claim 3, wherein the processor is configured to set the determined depth.

7. The ultrasound diagnostic apparatus according to claim 4, further comprising an interface through which the user performs an input operation,
wherein the processor is configured to set, as the determined depth, a depth input by the user through the interface.

8. The ultrasound diagnostic apparatus according to claim 7, wherein the processor is configured to set, as the determined depth, a depth at a position designated by the user through the interface in the ultrasound image.

9. The ultrasound diagnostic apparatus according to claim 4, wherein the processor is configured to perform image analysis on the ultrasound image to detect an inhibition site into which an entry of the insertion device is inhibited.

10. The ultrasound diagnostic apparatus according to claim 9, further comprising:
an interface through which the user performs an input operation,
wherein the processor is configured to
present a plurality of depth candidates related to the determined depth to the user on the basis of the inhibition site, and
set the determined depth from a depth candidate selected by the user from among the plurality of depth candidates through the interface.

11. The ultrasound diagnostic apparatus according to claim 9, wherein the processor is configured to set, as the determined depth, a depth at a shallowest position within an area occupied by the inhibition site.

12. The ultrasound diagnostic apparatus according to claim 9, wherein the processor is configured to
update the ultrasound image each time reception of an ultrasound echo along a predetermined number of scan lines is performed by the array transducer, and
detect the inhibition site in the ultrasound image which is updated.

13. The ultrasound diagnostic apparatus according to claim 12, wherein the processor is configured to update the determined depth on the basis of an area occupied by the inhibition site each time the ultrasound image is updated.

14. The ultrasound diagnostic apparatus according to claim 9, wherein the processor is configured to
control the transmission of the ultrasound beams to perform a pre-scan of the subject, and
perform the image analysis on the ultrasound image obtained by the pre-scan.

15. The ultrasound diagnostic apparatus according to claim 1, wherein the processor is configured to provide the notification to the user by at least one of generating a warning sound or vibrating the probe.

16. The ultrasound diagnostic apparatus according to claim 1, further comprising a display that displays the ultrasound image,
wherein the processor is configured to provide the notification to the user by providing a warning display on the display.

17. The ultrasound diagnostic apparatus according to claim 16, wherein the processor is configured to provide the warning display by changing a color of a tip portion of the insertion device to be displayed on the display in accordance with a difference between the insertion depth of the insertion device and the determined depth.

18. The ultrasound diagnostic apparatus according to claim 1, further comprising a display that displays the ultrasound image,
wherein the processor is configured to provide the notification to the user by freezing the ultrasound image on the display.

19. The ultrasound diagnostic apparatus according to claim 1, wherein the insertion device is a puncture needle, a catheter, or forceps.

20. A method for controlling an ultrasound diagnostic apparatus, comprising:
transmitting and receiving respective ultrasound beams to and from a subject along a plurality of scan lines;
emitting light to an insertion device insertable into the subject and having a photoacoustic wave generation unit;
receiving a photoacoustic wave generated from the photoacoustic wave generation unit in response to the photoacoustic wave generation unit being irradiated with the emitted light;
controlling transmission and reception of the ultrasound beams, emission of light to the insertion device, and reception of the photoacoustic wave so that the photoacoustic wave is received each time an ultrasound echo is received along a predetermined number of scan lines;
detecting an insertion depth of the insertion device on the basis of a signal of the photoacoustic wave that is received; and
providing a notification to a user in a case where the detected insertion depth of the insertion device is deeper than a determined depth.
